(19) United States
(12) Patent Application Publication
(10) Pub. No.: US 2006/0099259 A1
(43) Pub. Date: May 11, 2006

(54) Propranolol formulations

(76) Inventor: Grant Heinicke, Rockaway, NJ (US)

Correspondence Address: CANTOR COLBURN, LLP, 55 GRIFFIN ROAD SOUTH, BLOOMFIELD, CT, 06002, US

(21) Appl. No.: 10/983,202

(22) Filed: Nov. 5, 2004

(51) Int. Cl. A61K 31/138; A61K 9/24

(52) U.S. Cl. 424/472; 514/651

(57) ABSTRACT

Controlled-release propranolol formulations comprise a core comprising a pharmaceutically acceptable propranolol salt and an inert core; and a coating disposed on the core, the coating comprising about 70:30 to about 85:15 of ethylcellulose:polyvinylpyrrolidone or about 60:40 to about 75:25 of ethylcellulose:hydroxypropylmethylcellulose.

### BACKGROUND

Propranolol [1-(isopropyl amino)-3-(1-naphthyloxy)-2-propanol] is a beta-adrenergic blocking agent and as such is a competitive inhibitor of the effects of catecholamines at beta-adrenergic receptor sites. The principal effect of propranolol is to reduce cardiac activity by diminishing or preventing beta-adrenergic stimulation. By reducing the rate and force of contraction of the heart, and decreasing the rate of conduction of impulses through the conducting system, the response of the heart to stress and exercise is reduced. These properties are used in the treatment of angina in an effort to reduce the oxygen consumption and increase the exercise tolerance of the heart. Propranolol is also used in the treatment of cardiac arrhythmias to block adrenergic stimulation of cardiac pacemaker potentials. Propranolol is also beneficial in the long-term treatment of hypertension. Other uses of propranolol are in the treatment of migraine and anxiety.

The present invention addresses the need for improved propranolol dosage forms, particularly controlled-release dosage forms.

### SUMMARY

In one embodiment, a composition comprises a core comprising a pharmaceutically acceptable propranolol salt disposed on an inert core; and a coating disposed on the core, the coating comprising about 70:30 to about 85:15 of ethylcellulose:polyvinylpyrrolidone, controlled-release or about 60:40 to about 75:25 of ethylcellulose:hydroxypropylmethylcellulose.

In another embodiment, a dosage form comprises a core comprising a pharmaceutically acceptable propranolol salt disposed on an inert core; and a coating disposed on the core, the coating comprising hydroxypropylmethylcellulose, polyvinylpyrrolidone, or a combination thereof; and ethylcellulose; wherein the dosage form comprises one type of coated core; and wherein the average Cₘₐₓ of the dosage form is about 105 ng/mL to about 270 ng/mL and the average AUC_{0-∞} of the dosage form is about 2100 ng hr/mL to about 5400 ng hr/mL when measured under fasting conditions, or wherein the average Cₘₐₓ of the dosage form is about 70 ng/mL to about 216 ng/mL and the average AUC_{0-∞} of the dosage form is about 1400 ng hr/mL to about 4725 ng hr/mL when measured under fed conditions.

Also included is a method of treating a human comprising administering a pharmaceutically effective amount of the disclosed dosage forms to a human in need of treatment for angina, cardiac arrhythmia, or hypertension.

These and other embodiments, advantages and features of the present invention become clear when detailed description and examples are provided in subsequent sections.

### DETAILED DESCRIPTION

### Chemical Description and Terminology

The use of the terms “a” and “an” and “the” and similar referents (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention as used herein, the terms wt %, weight percent, percent by weight, etc. are equivalent and interchangeable.

The term “active agent” is meant to include solvates (including hydrates) of the free compound or salt, crystalline and non-crystalline forms, as well as various polymorphs. Unless otherwise specified, the term “active agent” is used herein to indicate propranolol or a pharmaceutically acceptable salt thereof. For example, an active agent can include all optical isomers of propranolol and all pharmaceutically acceptable salts thereof either alone or in combination.

“Pharmaceutically acceptable salts” includes derivatives of propranolol, wherein the propranolol is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of the propranolol. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

By “oral dosage form” is meant to include a unit dosage form prescribed or intended for oral administration. An oral dosage form may or may not comprise a plurality of subunits such as, for example, microcapsules or microtablets, packaged for administration in a single dose.

By “subunit” is meant to include a composition, mixture, particle, etc., that can provide an oral dosage form alone or when combined with other subunits. By “part of the same subunit” is meant to refer to a subunit comprising certain ingredients.

Dissolution profile as used herein, means a plot of the cumulative amount of active ingredient released as a function of time. The dissolution profile can be measured utilizing the Drug Release Test <724>, which incorporates standard test USP 26 (Test <711>). A profile is characterized by the test conditions selected. Thus the dissolution profile can be generated at a preselected apparatus type, shaft speed, temperature, volume, and pH of the dissolution media.

Release forms may also be characterized by their pharmacokinetic parameters. “Pharmacokinetic parameters” are parameters which describe the in vivo characteristics of the active agent over time, including for example the in vivo dissolution characteristics and plasma concentration of the active agent. By “Cₘₐₓ” is meant the measured concentration of the active agent in the plasma at the point of maximum concentration. By “C₂₄” is meant the concentration of the active agent in the plasma at about 24 hours. The term “Tₘₐₓ” refers to the time at which the concentration of the active agent in the plasma is the highest. “AUC” is the area under the curve of a graph of the concentration of the active agent (typically plasma concentration) vs. time, measured from one time to another.

By “instant-release” is meant a dosage form designed to ensure rapid dissolution of the active agent by modifying the normal crystal form of the active agent to obtain a more rapid dissolution. By “immediate-release”, it is meant a conventional or non-modified release in which greater then or equal to about 75% of the active agent is released within two hours of administration, preferably within one hour of administration.

By “controlled-release” it is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours.

Dosage forms can be combination dosage forms having both immediate release and controlled release characteristics, for example, a combination of immediate release pellets and controlled release pellets. The immediate release portion of the dosage form may be referred to as a loading dose.

Certain formulations described herein may be “coated”. The coating can be a suitable coating, such as, a functional or a non-functional coating, or multiple functional and/or non-functional coatings. By “functional coating” is meant to include a coating that modifies the release properties of the total formulation, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

Dosage Forms

In one embodiment, unit dosage forms of controlled-release propranolol hydrochloride are provided that will release the drug into an aqueous environment in a manner similar to that of INDERAL® LA when tested under in vitro and/or in vivo conditions. In some embodiments, the unit dosage forms may have similar behavior to INDERAL® LA in vitro but not in vivo, and vice versa. The controlled-release dosage forms may be optimized to obtain release profiles similar to that of INDERAL® LA, when both the reference product and the dosage forms described herein are tested by the United States Pharmacopoeia method for Propranolol Hydrochloride Extended Release Capsules. The disclosed unit dosage forms may be bioequivalent to INDERAL® LA when compared on an mg-by-mg basis. In addition, the dosage forms described herein may be physically and chemically stable dosage forms (i.e., exhibiting drug release profiles and degradation profiles statistically similar to that at the initial time point) when subjected to stability studies.

A controlled-release propranolol dosage form comprises a core comprising a propranolol-coated inert sphere, wherein the core is coated with a controlled-release coating composition comprising hydroxypropylmethylcellulose, polyvinylpyrrolidone, or a combination thereof, and ethylcellulose. It has been unexpectedly found by the inventors herein that particular controlled-release coated spheres can be employed to provide dosage forms having bioavailability comparable to INDERAL® LA. In one embodiment, the coated spheres can be used as a single type of subunit in a dosage form. In another embodiment, the controlled-release coated spheres can be one of two or more types of subunits in a dosage form.

The controlled-release propranolol formulation is based on pellets having an inert core component. Suitable inert cores include for example, sugar spheres, particulate microcrystalline cellulose, silicon dioxide spheres, wax beads such as prilled waxes, and combinations comprising one or more of the foregoing inert cores. In one embodiment, the core comprises non-pareil sugar seeds (sugar spheres, USP XXII) having an average size of about 25 to about 35 mesh (500 to 710 micrometers), or about 25 to about 30 mesh (600 to 710 micrometers), or about 30 to about 35 mesh (500-600 micrometers). A composition comprising propranolol (e.g., propranolol hydrochloride) is disposed on the inert core in an amount sufficient to provide a dosage form comprising about 20 to about 200 mg of propranolol hydrochloride (e.g., 60 mg, 80 mg, 120 mg, and 160 mg).

The cores (e.g., sugar spheres containing propranolol) are then coated with a controlled-release coating composition that provides for the controlled release of propranolol. The controlled-release coating composition is applied (e.g., by spraying) the cores to form a controlled-release coating disposed on the cores. In one embodiment, the controlled-release coating composition comprises hydroxypropylmethylcellulose, polyvinylpyrrolidone, or a combination thereof; and ethylcellulose. The ratio of ethylcellulose to hydroxypropylmethylcellulose, polyvinylpyrrolidone, or a combination thereof is 60:40 to 85:15. When the coating comprises hydroxypropylmethylcellulose, the ratio of ethylcellulose to hydroxypropylmethylcellulose may be 60:40 to 75:25. When the coating comprises polyvinylpyrrolidone, the ratio of ethylcellulose to polyvinylpyrrolidone may be 70:30 to 85:15, or 75:25 to 80:20.

The components of the controlled-release coating may be characterized by their viscosity measured as a 2% solution in 20:80 ethanol:toluene at 20° C. A suitable form of ethylcellulose is that having a viscosity of about 5 cps to about 20 cps at 20° C. In one embodiment, the ethylcellulose has a viscosity of about 12 cps to about 16 cps at 20° C. A suitable form of hydroxypropylmethylcellulose is that having a viscosity of 5 to 18 at 20° C., or 15 cps at 20° C. A suitable form of polyvinylpyrrolidone is that having a viscosity of 5.5 to 8.5 cps at 20° C. The coating may optionally comprise a plasticizer, for example a vegetable oil, for example castor oil, or glycerol; a glyceryl ester of a fatty acid, for example glyceryl triacetate or glyceryl monoricinoleate; dibutyl sebacate; triethyl citrate; acetyl triethyl citrate; tributyl citrate; acetyl tributyl citrate; diethyl phthalate; dimethyl phthalate; and combinations comprising one or more of the foregoing plasticizers. In addition, the coating may comprise a processing agent, such as, for example, talc, kaolin, silicon dioxide, magnesium stearate, and combinations comprising one or more of the foregoing processing aids. Suitable amounts of the plasticizer and processing aid can be readily determined by one of skill in the art.

The controlled-release coating composition also comprises a solvent to facilitate application of the composition to the cores. Suitable solvents include, for example, water; and alcohols such as ethanol, denatured ethanol, and methanol; and combinations comprising one or more of the foregoing solvents. In some cases, it may be desirable to add methylene chloride to the coating composition which can aid in preventing the polymers from settling out of the coating composition

The controlled-release coating composition can be applied to the core using a coating technique used in the pharmaceutical industry, such as fluid bed coating. Once applied, the controlled-release coating may comprise 10 wt % to about 17 wt % of the total weight of the coated cores, or about 12 wt % to about 15 wt % of the total weight of the coated cores.

The controlled-release coating may be dried before applying an optional second coating. A color imparting agent may be added to the controlled-release coating composition or a rapidly dissolving seal coat containing color may be coated over the controlled release coating layer provided that the seal coat is compatible with and does not affect the dissolution of the controlled-release coating layer.

In one embodiment, the coated core is free from added organic acid, such as, for example, citric acid, tartaric acid, succinic acid, malic acid, ascorbic acid, and fumaric acid.

The dosage form optionally comprises a loading dose of propranolol. The loading dose comprises 0 percent by weight (wt %) to about 30 wt %, more specifically 0 wt % to about 15 wt % of the total weight of the combination of the loading dose and the coated cores. The loading dose may be, for example, in the form of an immediate-release subunit. The loading dose may comprise suitable excipients as are known in the art so long as the release characteristics of the loading dose are not affected.

The coated cores and optional loading dose may be placed in a gelatin capsule or they may be made into tablets, for example, by first adding about 25 wt % to about 40 wt % of a solid pharmaceutically acceptable tablet excipient which will form a compressible mixture with the coated cores and which may be formed into a tablet without crushing the coated cores, and optionally an effective amount of a tablet disintegrating agent and a lubricant. The solid pharmaceutically acceptable tablet excipient may comprise, for example, lactose, dextrose, mannitol, calcium phosphate, microcrystalline cellulose, powdered sucrose, or combinations comprising one or more of the foregoing excipients. The tablet disintegrant may comprise crospovidone, croscarmellose sodium, dry starch, sodium starch glycolate, and the like, and combinations comprising one or more of the foregoing disintegrants. Suitable lubricants include, for example, calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants.

The dosage forms disclosed herein may exhibit an in vitro dissolution profile substantially corresponding to the following pattern when tested according to a proposed USP method for propranolol in which dissolution is measured either in 0.1 N HCl or at pH 6.8 in a USP apparatus. In one embodiment, the coated cores have the following in vitro release profile when dissolution is performed in a pH 6.8 medium:
less than 10 wt % of the propranolol released at 1 hour;
39 wt % to 69 wt % of the propranolol released at 6 hours; and
greater than 80 wt % of the propranolol released at 15 hours.

In another embodiment, the coated cores have the following in vitro release profile when dissolution is performed in a pH 6.8 medium:
less than 10 wt % of the propranolol released at 1 hour;
44 wt % to 64 wt % of the propranolol released at 6 hours; and
greater than 80 wt % of the propranolol released at 15 hours.

In yet another embodiment, the coated cores have the following in vitro release profile when dissolution is performed in 0.1 M HCl:
less than 10 wt % of the propranolol released at 1 hour;
35 wt % to 65 wt % of the propranolol released at 6 hours; and
greater than 80 wt % of the propranolol released at 15 hours.

In another embodiment, the coated cores have the following in vitro release profile when dissolution is performed in 0.1 M HCl:
less than 10 wt % of the propranolol is released after 1 hour;
40 wt % to 60 wt % of the propranolol is released after 6 hours; and
greater than 80 wt % of the propranolol is released after 15 hours.

The controlled-release core described herein can be formulated into propranolol dosage forms that are bioequivalent to INDERAL® LA. In one embodiment, the disclosed controlled-release compositions have an average maximum blood plasma concentration (Cₘₐₓ) of 70% to 135% of that of INDERAL® LA and an average AUC_{0-∞} of 70% to 135% of that of INDERAL® LA. In one embodiment, the average Cₘₐₓ for INDERAL® LA when measured in the fasting mode is about 150 ng/mL to about 200 ng/mL, and the average AUC_{0-∞} when measured in the fasting mode is about 3000 ng hr/mL to about 4000 ng hr/mL. Thus, when measured in the fasting mode, the average Cₘₐₓ of the dosage form may be about 105 ng/mL to about 270 ng/mL and the average AUC_{0-∞} about 2100 ng hr/mL to about 5400 ng hr/mL. In another embodiment, the average Cₘₐₓ for INDERAL® LA when measured in the fed mode is about 100 ng/mL to about 160 ng/mL, and the average AUC_{0-∞} when measured in the fed mode is about 2000 ng hr/mL to about 3500 ng hr/mL. Thus, when measured in the fed mode, the average Cₘₐₓ of the dosage form may be about 70 ng/mL to about 216 ng/mL and the average AUC_{0-∞} about 1400 ng hr/mL to about 4725 ng hr/mL.

### EXAMPLE 1

### Formulation of a First Dosage Form

Cores comprising propranolol hydrochloride were formed by first mixing 11,390 g of propranolol hydrochloride, 19,975 g of a granulating solution comprising 799 grams of hydroxypropyl cellulose NF (75-150 cps), and denatured alcohol to form a suspension. This suspension was sprayed onto 4,811 g of sugar spheres (500 μm to 600 μm) at a flow rate of 180 g/min to 340 g/min and a product temperature of 11° C. to 20° C. Spraying was continued until all of the suspension was coated. The cores formed weighed about 17,000 g.

The cores were then spray coated with a controlled release coating composition comprising 8,470 g ethylcellulose (Aqualon N14 Pharm), 2,530 g Povidone USP (Plasdone K 29/32), and 209,000 g denatured alcohol. 53 kg of cores was sprayed with 158,300 g of the coating composition. After coating was complete the coated cores were dried at a temperature of 50° C.

The dissolution profile of the coated core was measured in 0.1 N HCl.

TABLE 1

| Time (minutes) | Percent release |
|---|---|
| 60 | 2.3 |
| 120 | 12.9 |
| 240 | 33.6 |
| 360 | 53.1 |
| 480 | 68.6 |
| 720 | 85.3 |
| 900 | 90.9 |
| 1080 | 94.1 |

### EXAMPLE 2

### Formulation of a Second Dosage Form

Cores comprising propranolol hydrochloride were formed by first mixing 1751 g of propranolol hydrochloride, 123 g hydroxypropyl cellulose NF (75-150 cps), and 3075 g denatured alcohol to form a suspension. This suspension was sprayed onto 830 g of microcrystalline cellulose spheres (350 μm to 360 μm). Spraying was continued until all of the suspension was coated. The cores formed weighed about 2,679 g. Onto 770 grams of cores, 1385 grams of controlled-release coating composition as in Example 1 was sprayed.

The dissolution profile of the coated core was measured in 0.1 N HCl.

TABLE 2

| Time (minutes) | Percent release |
|---|---|
| 60 | 2.3 |
| 120 | 11 |
| 240 | 34 |
| 360 | 55 |
| 480 | 70 |
| 720 | 83 |
| 900 | 88 |
| 1080 | 91 |

### EXAMPLE 3

### Biostudy

A randomized, single-dose, three-way crossover pilot study design may be used to evaluate the relative bioavailability of the propranolol controlled-release capsules when dosed (1×160 mg) under fasting conditions.

Sixteen (16) subjects healthy adults may be recruited for the study. Propranolol concentrations in plasma may be determined by a validated LC/MS/MS method. Subjects were assayed for propranolol.

The following pharmacokinetic parameters may be determined from the plasma concentration data:

The area under the plasma concentration versus time curve [AUC_{(0-TLQC)}] may be calculated using the linear trapezoidal rule from the zero time point to the last quantifiable concentration. AUC_{(0-TLQC)} may also be designated as AUCTLQC.

The area under the plasma concentration versus time curve from zero to infinity [AUC_{(0-INF)}] may be calculated by adding Cₜ/Kₑₗₘ to AUC_{(0-TLQC)} where Cₜ is the last quantifiable concentration and Kₑₗₘ is the elimination rate constant. The AUC_{(0-INF)} may also be designated as AUCINF.

The maximum observed plasma concentration [Cₘₐₓ] may be obtained by inspection. The Cₘₐₓ may also be designated as CMAX.

The time to maximum plasma concentration [Tₘₐₓ] may be obtained by inspection. If the maximum plasma concentration occurs at more than one time point, the first may be chosen as TMAX. The Tₘₐₓ may also be designated as TMAX.

The terminal elimination rate constant [Kₑₗₘ] may be obtained from the slope of the line, fitted by linear least squares regression, through the terminal points of the log(base e) of the concentration versus time plot for these points. The Kₑₗₘ may also be designated as KELM.

The half-life [T_{1/2}] may be calculated by the equation T_{1/2}=0.693/Kₑₗₘ. The T_{1/2} may also be designated as THALF.

The elimination of drug from the plasma may be polyphasic for some of the subjects. The elimination rate constants may be estimated from the plasma data for all subjects using the plasma concentrations of the elimination phase as best as can be determined from the plasma propranolol concentration vs time plots (log scale) for the individual subjects. In some cases, the elimination phase may not be well characterized. No elimination rate constant (KELM) is reported for these cases, and also no values for AUCINF and THALF are reported.

Most plasma samples may be collected near the target times. Corrections may be made in the calculations for any blood draw deviations that are more than ±5% from the target times as reported by the clinic.

Statistical analyses appropriate for a three-period crossover design may be performed to assess the bioequivalence of the three products when dosed. The analyses may be performed using SAS® software. The calculations for the 90% confidence interval about the ratio of the mean test value to mean reference value and for the power of the ANOVA to detect a 20% difference from the reference mean may be performed using the LSMEAN values and standard error of estimate values as generated by the SAS software. The ratios of geometric means and the 90% confidence intervals of the log (base e) transformed data may be calculated for AUCTLQC, AUCINF, and CMAX.

Novel controlled-release propranolol dosage forms comprising coated spheres have been described. The dosage forms may advantageously be bioequivalent to the commercially available INDERAL® LA.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising:
a core comprising a pharmaceutically acceptable propranolol salt disposed on an inert core; and
a coating disposed on the core, the coating comprising about 70:30 to about 85:15 of ethylcellulose:polyvinylpyrrolidone, or about 60:40 to about 75:25 of ethylcellulose:hydroxypropylmethylcellulose.

2. The composition of claim 1, wherein the inert core comprises a sugar sphere having a diameter of about 500 to about 710 micrometers.

3. The composition of claim 1, wherein the coating comprises 10 wt % to about 17 wt % of the total weight of the coated cores.

4. The composition of claim 1, wherein the coating comprises about 12 wt % to about 15 wt % of the total weight of the coated cores.

5. The composition of claim I, wherein the coating comprises ethylcellulose having a viscosity of 5 cps to about 20 cps at 20° C. and polyvinylpyrrolidone having a viscosity of about 5.5 to 8.5 cps at 20° C.

6. The composition of claim 1, wherein the coating comprises ethylcellulose having a viscosity of 5 cps to about 20 cps at 20° C. and hydroxypropylmethylcellulose having a viscosity of about 5 to about 18 cps at 20° C.

7. The composition of claim 1, exhibiting a dissolution profile in a pH 6.8 medium such that:
less than 10 wt % of the propranolol is released at 1 hour;
39 wt % to 69 wt % of the propranolol is released at 6 hours; and
greater than 80 wt % of the propranolol is released at 15 hours.

8. The composition of claim 1, exhibiting a dissolution profile in a pH 6.8 medium such that:
less than 10 wt % of the propranolol is released at 1 hour;
44 wt % to 64 wt % of the propranolol is released at 6 hours; and
greater than 80 wt % of the propranolol is released at 15 hours.

9. The composition of claim 1, exhibiting a dissolution profile in 0.1 M HCl such that:
less than 10 wt % of the propranolol is released at 1 hour;
35 wt % to 65 wt % of the propranolol is released at 6 hours; and
greater than 80 wt % of the propranolol is released at 15 hours.

10. The composition of claim 1, exhibiting a dissolution profile in 0.1 M HCl such that:
less than 10 wt % of the propranolol is released at 1 hour;
40 wt % to 60 wt % of the propranolol is released at 6 hours; and
greater than 80 wt % of the propranolol is released at 15 hours.

11. The composition of claim 1, wherein the coated core comprises no added organic acid.

12. A dosage form comprising:
a core comprising a pharmaceutically acceptable propranolol salt disposed on an inert core; and
a coating disposed on the core, the coating comprising hydroxypropylmethylcellulose, polyvinylpyrrolidone, or a combination thereof; and ethylcellulose;
wherein the dosage form comprises one type of controlled-release coated core; and
wherein the average Cₘₐₓ of the dosage form is about 105 ng/mL to about 270 ng/mL and the average AUC_{0-∞} of the dosage form is about 2100 ng hr/mL to about 5400 ng hr/mL when measured under fasting conditions, or
wherein the average Cₘₐₓ of the dosage form is about 70 ng/mL to about 216 ng/mL and the average AUC_{0-∞} of the dosage form is about 1400 ng hr/mL to about 4725 ng hr/mL when measure under fed conditions.

13. The dosage form of claim 12, wherein the coating comprises about 70:30 to about 85:15 of ethylcellulose:polyvinylpyrrolidone.

14. The dosage form of claim 12, wherein the coating comprises about 60:40 to about 75:25 of ethylcellulose:hydroxypropylmethylcellulose.

15. The dosage form of claim 12, wherein the ethylcellulose has a viscosity of 5 cps to about 20 cps at 20° C. and the polyvinylpyrrolidone has a viscosity of about 5.5 to 8.5 cps at 20° C.

16. The dosage form of claim 12, wherein the coating comprises about 60 wt % to about 75 wt % of ethylcellulose and about 25 wt % to about 40 wt % of hydroxypropylmethylcellulose.

17. The dosage form of claim 16, wherein the coating comprises ethylcellulose having a viscosity of 5 cps to about 20 cps at 20° C. and hydroxypropylmethylcellulose having a viscosity of about 5 to about 18 cps at 20° C.

18. The dosage form of claim 12, wherein the coated core comprises no added organic acid.

19. The dosage form of claim 12, wherein the dosage form comprises a capsule.

20. A method of treating a human, comprising administering a pharmaceutically effective amount of the dosage form of claim 13 to a human in need of treatment for angina, cardiac arrhythmia, or hypertension.

21. The method of claim 20, wherein the coated core comprises no added organic acid.

22. The method of claim 20, wherein the dosage form comprises a capsule.
